(19) United States
(12) Patent Application Publication
(10) Pub. No.: US 2018/0244600 A1
(43) Pub. Date: Aug. 30, 2018

(54) Purification of 3,6-dichlorosalicylic acid using selective solubilization of impurities

(71) Applicants: Bomi P. Framroze, Stanford, CA (US); Rajagopal Kuppuswamy, Chennai (IN); Rajaiah Srikrishnan, Kanchipuram (IN)

(72) Inventors: Bomi P. Framroze, Stanford, CA (US); Rajagopal Kuppuswamy, Chennai (IN); Rajaiah Srikrishnan, Kanchipuram (IN)

(21) Appl. No.: 15/393,273

(22) Filed: Dec. 29, 2016

(51) Int. Cl. C07C 51/43; C07C 65/10

(52) U.S. Cl. CPC C07C 51/43; C07C 65/10

(57) ABSTRACT

A process to purify 3,6-dichlorosalicylic acid by selectively solubilizing impurities and unreacted starting materials without loss of yield.

### Field

This invention relates to a new process for purification of 3,6-dichlorosalicylic acid from its undesired isomer 4,5-dichlorosalicylic acid as well as from any unreacted dichlorophenol starting materials by the use of an chloroalkane solvent to selectively solubilize the undesired compounds.

3,6-dichlorosalicylic acid is an important intermediate in the manufacture of pharmaceuticals and agrochemicals, particularly the herbicide, Dicamba. The process of this invention directly results in the manufacture of greater than 99% purity 3,6-dichlorosalicylic acid. Further, the described process leads to a more commercially viable overall process to manufacture dicamba, eliminating the need to use 99% 2,5-dichlorophenol or 2,5-dichloroaniline and allowing for the use of the much cheaper 75% to 80% crude 2,5-dichlorophenol instead.

### BACKGROUND

3,6-dichlorosalicylic acid is an important intermediate in the agrochemical industry. The ability for a general, efficient synthesis of 3,6-dichlorosalicylic acid with greater than 99% purity is still of continuing interest to the industry. The two main routes described are the route from 2,5-dichloroaniline and the route from 1,2,4-trichlorobenzene. The route from 99% 2,5-dichloroaniline proceeds via diazotization and hydrolysis to give 99% 2,5-dichlorophenol which is carboxylated under prior art described Kolbe conditions to give 99% 3,6-dichlorosalicylic acid. However, the starting 99% 2,5-dichloroaniline is difficult to make, has low yields and is thus expensive, making this route commercially unsuitable.

The second route from 1,2,4-trichlorobenzene starts with the much cheaper raw material and base hydrolysis with sodium hydroxide results in a mixture of 2,3-dichlorophenol, 2,4-dichlorophenol and 75% to 80% 2,5-dichlorophenol.This final mixture of products needs to be fractionally distilled at high vacuum and large number of theoretical plates to arrive at the desired 99% pure 2,5-dichlorophenol for further carboxylation as described above. The high energy cost and lowered hydrolysis yields also makes it commercially undesirable. One alternate to directly hydrolyzing 1,2,4-trichlorobenzene is to make the benezensulfonic acid analog first and hydrolyze this in a much less dangerous manner under atmospheric pressure but this method although safer, still results in 92% to 94% 2,5-dichlorophenol only.

Recently more efforts have been made for the development of novel processes to arrive at greater than 99% pure 3,6-dichlorosalicylic acid. For example, as described in GB2341178, using selective crystallization from its eutectic mixture to increase the concentration of 2,5-dichlorophenol from the mixture of dichlorophenols but the commercial yields and reproducibility have not been proven. Other researchers have tried to selectively oxidize 1,4-dichlorobenzene directly to 99% isomerically pure 2,5-dichlorophenol using vanadium catalysts as described in U.S. Pat. No. 6,323,377 and U.S. Pat. No. 6,586,624 but the catalyst consumption is not commercially viable.

None of the described methods of the prior art show any method to directly carboxylate a less than 98% pure 2,5-dichlorophenol mixture and use a cleanup step on the crude carboxylated product to arrive at greater than 99% 3,6-dichlorosalicylic acid. In particular, no prior art describes the use of any solvent to selectively solubilize both the undesired impurities, 4,5-dichlorosalicylic acid and 2,5-dichlorophenol, to arrive at greater than 99% pure 3,6-dichlorosalicylic acid.

Accordingly, there remains a need to devise a route to prepare greater than 99% pure 3,6-dichlorosalicylic acid directly after the carboxylation of a mixture of 90% to 95% dichlorophenols arrived at after hydrolysis of 1,2,4-trichlorobenzene in a cost effective and environmentally acceptable manner.

### SUMMARY OF THE INVENTION

The present invention describes a method to produce greater than 99% pure 3,6-dichlorosalicylic acid after carboxylating 90% to 95% 2,5-dichlorophenol and selectively solubilizing the undesired 4,5-dichlorosalicylic acid and unreacted 2,5-dichlorophenol using an alkyl dichloride solvent wash of the crude filter cake arrived at after work up of the carboxylation reaction. This method eliminates the use of dangerous reagents and high-energy distillation columns and in a simple and easy manner makes greater than 99% pure 3,6-dichlorosalicylic acid.

### DETAILED DESCRIPTION OF THE INVENTION

Before describing the present invention in detail, it is to be understood that the terminology used herein is for the purpose of describing particular embodiments only, and is not intended to be limiting. More specifically, as utilized herein, the term “alkyl”, alone or in combination, means a straight-chain or branched-chain alkyl radical containing from 1 to about 10, preferably from 1 to about 8, carbon atoms. Examples of such radicals include methyl, ethyl, n-propyl, n-butyl, isobutyl, sec-butyl, tert-butyl, pentyl, isoamyl, hexyl, octyl and the like.

In accordance with the present invention, there is provided a process to arrive at the agrochemical herbicide intermediate 3,6-dichlorosalicylic acid in greater than 99% purity in an environmentally safe and cost effective manner.

For the purpose of the following examples on purification of 3,6-dichlorosalicylic acid, a standard preparation of crude 3,6-dichlorosalicylic acid was used. A 93% 2,5-dichlorophenol powder was carboxylated as described in the prior art (Kolbe-Schmidt reaction). After completion of the carboxylation reaction, the xylene solvent used was recovered by vacuum distillation and acidified with hydrochloric acid. The resultant slurry was filtered and dried under vacuum for 12 hours. Analysis of this crude 3,6-dichlorosalicylic acid product thus produced shows by

GC area percent analysis to be:

3,6-dichlorosalicylic acid: 68.87%

4,5-dichlorosalicylic acid: 3.65%

2,5-dichlorophenol: 26.87%

Others unidentified peaks: 0.61%

This crude mixture was subjected to a purification procedure using a variety of solvents as shown in the Examples below to remove the undesired 4,5-dichlorosalicyclic acid isomer and the starting 2,5-dichlorophenol and arrive at the desired greater than 99% purity of 3,6-dichlorosalicylic acid.

### EXAMPLE 1

### Purification of 3,6-dichlorosalicylic Acid by Washing Filter Cake with Ethylene Dichloride Solvent.

100 g of the crude 3,6-dichlorosalicylic acid cake was added to a stirring flask and 100 ml of ethylene dichloride was added to it. The mixture was stirred for 1 hr at 50° C. to 55° C. and vacuum filtered. The procedure was repeated again and the resultant powder dried under vacuum for 12 hours to give 84 g of free flowing white powder. The resulting powder was analyzed and showed by GC area percent analysis to be:

3,6-dichlorosalicylic acid: 99.5%

4,5-dichlorosalicylic acid: 0.1%

2,5-dichlorophenol: 0.3%

Other unidentified peaks: 0.1%

The analysis profile of mother liquor (filtrate) showed by GC area percent analysis to be

3,6-dichlorosalicylic acid: 8.8%

4,5-dichlorosalicylic acid: 10.9%

2,5-dichlorophenol: 79.3%

Other unidentified peaks: 1.0%

### EXAMPLE 2

### Purification of 3,6-dichlorosalicylic Acid by Washing Filter Cake with Xylene.

100 g of the crude 3,6-dichlorosalicylic acid cake was added to a stirring flask and 100 ml of xylene was added to it. The mixture was stirred for 1 hr at 90° C. and vacuum filtered. The procedure was repeated again and the resultant powder dried under vacuum for 12 hours to give 72 g of light yellow powder. The resulting powder was analyzed and showed by GC area percent analysis to be:

3,6-dichlorosalicylic acid: 95.8%

4,5-dichlorosalicylic acid: 0.6%

2,5-dichlorophenol: 3.2%

Other unidentified peaks: 0.4%

The analysis profile of mother liquor (filtrate) showed by GC area percent analysis to be

3,6-dichlorosalicylic acid: 13.8%

4,5-dichlorosalicylic acid: 10.0%

2,5-dichlorophenol: 69.2%

Others: 7.0%

### EXAMPLE 3

### Purification of 3,6-dichlorosalicylic Acid by Washing Filter Cake with Toluene.

100 g of the crude 3,6-dichlorosalicylic acid cake was added to a stirring flask and 100 ml of toluene was added to it. The mixture was stirred for 1 hr at 90° C. and vacuum filtered. The procedure was repeated again twice and the resultant powder dried under vacuum for 12 hours to give 70 g of light yellow powder. The resulting powder was analyzed and showed by GC area percent analysis to be:

3,6-dichlorosalicylic acid: 96.0%

4,5-dichlorosalicylic acid: 0.4%

2,5-dichlorophenol: 3.1%

Other unidentified peaks: 0.5%

The analysis profile of mother liquor (filtrate) showed by GC area percent analysis to be

3,6-dichlorosalicylic acid: 15.5%

4,5-dichlorosalicylic acid: 10.2%

2,5-dichlorophenol: 73.5%

Others: 0.8%

### EXAMPLE 4

### Purification of 3,6-dichlorosalicylic Acid by Washing Filter Cake with Hexane.

100 g of the crude 3,6-dichlorosalicylic acid cake was added to a stirring flask and 100 ml of hexane was added to it. The mixture was stirred for 1 hr at 50° C. and vacuum filtered. The procedure was repeated again twice and the resultant powder dried under vacuum for 12 hours to give 90 g of light yellow powder. The resulting powder was analyzed and showed by GC area percent analysis to be:

3,6-dichlorosalicylic acid: 95.2%

4,5-dichlorosalicylic acid: 3.6%

2,5-dichlorophenol: 1.1%

Other unidentified peaks: 0.1%

The analysis profile of mother liquor (filtrate) showed by GC area percent analysis to be

3,6-dichlorosalicylic acid: 1.4%

4,5-dichlorosalicylic acid: nil

2,5-dichlorophenol: 91.5%

Others: 7.1%

### EXAMPLE 5

### Purification of 3,6-dichlorosalicylic Acid by Washing Filter Cake with Cyclohexane.

100 g of the crude 3,6-dichlorosalicylic acid cake was added to a stirring flask and 100 ml of cyclohexane was added to it. The mixture was stirred for 1 hr at 65° C. and vacuum filtered. The procedure was repeated again twice and the resultant powder dried under vacuum for 12 hours to give 87g of light yellow powder. The resulting powder was analyzed and showed by GC area percent analysis to be:

3,6-dichlorosalicylic acid: 94.6%

4,5-dichlorosalicylic acid: 3.6%

2,5-dichlorophenol: 1.7%

Other unidentified peaks: 0.1%

The analysis profile of mother liquor (filtrate) showed by GC area percent analysis to be

3,6-dichlorosalicylic acid: 1.6%

4,5-dichlorosalicylic acid 0.2%

2,5-dichlorophenol: 92.8%

Others: 5.4%

### EXAMPLE 6

### Purification of 3,6-dichlorosalicylic Acid by Washing Filter Cake with Ethyl Acetate.

100 g of the crude 3,6-dichlorosalicylic acid cake was added to a stirring flask and 100 ml of ethyl acetate was added to it. The cake got completely dissolved in the solvent at 40° C. Solubility of the cake was then investigate in ethyl acetate at 10° C. and 25° C. and was found to be greater than 40 wt % in both cases. Ethyl acetate is not suitable for washing the crude 3,6-dichlorosalicylic acid filter cake.

### EXAMPLE 7

### Purification of 3,6-dichlorosalicylic Acid by Washing Filter Cake with Dichloromethane Solvent.

100 g of the crude 3,6-dichlorosalicylic acid cake was added to a stirring flask and 100 ml of dichloromethane was added to it. The mixture was stirred for 1 hr at reflux temperature (38-42° C.) and vacuum filtered. The procedure was repeated again and the resultant powder dried under vacuum for 12 hours to give 68 g of free flowing white powder. The resulting powder was analyzed and showed by GC area percent analysis to be:

3,6-dichlorosalicylic acid: 99.25%

4,5-dichlorosalicylic acid: 0.4%

2,5-dichlorophenol: 0.36%

The analysis profile of mother liquor (filtrate) showed by GC area percent analysis to be

3,6-dichlorosalicylic acid: 36.6%

4,5-dichlorosalicylic acid: 13.5%

2,5-dichlorophenol: 49.1%

Other unidentified peaks: 0.8%

### EXAMPLE 8

### Purification of 3,6-dichlorosalicylic Acid by Washing Filter Cake with Solvent n-butyl chloride.

50 g of the crude 3,6-dichlorosalicylic acid cake was added to a stirring flask and 130 ml of n-butyl chloride was added to it. The mixture was stirred for 1 hr at reflux temperature (55-60° C.) and vacuum filtered. The procedure was repeated again and the resultant powder dried under vacuum for 12 hours to give 42 g of free flowing white powder. The resulting powder was analyzed and showed by HPLC area percent analysis to be:

3,6-DCSA: 99.03%

4,5-DCSA: 0.6%

2,5-DCP: 0.31%

The analysis profile of mother liquor (filtrate) showed by HPLC area percent analysis to be:

3,6-DCSA: 23.3%

4,5-DCSA: 22.86%

2,5-DCP: 40.15%

Other unidentified peaks: 13.69%

From the above results, it is clear that alkyl monochloro and dichloro solvents are the only solvents that yield greater than 99% purity of 3,6-dichlorosalicylic acid starting from 60% to 90% 3,6-dichlorosalicylic acid crude product.

It will be clear to those skilled in the art that modifications can be made to the process described herein without departing from the inventive concept set forth in our claims below.

What is claimed is:

1. A process to purify 3,6-dichlorosalicylic acid by selectively solubilizing all the undesired impurities and unreacted starting materials compounds from the lower purity 3,6-dichlorosalicylic acid in a chlorinated C1-C8 alkane solvent, filtering the resultant suspension and drying the wet cake to yield greater than 99% purity 3,6-dichlorosalicylic acid powder.

2. A process according to claim 1, wherein the starting purity of the 3,6-dichlorosalicylic acid is between 60% and 98%.

3. A process according to claim 1, wherein the C1-C8 alkane can be mono, di, or tri chloro substituted.

4. A process according to claim 1, wherein the preferred solvent is ethylene dichloride.

5. A process according to claim 1, wherein the ratio of the crude 3,6-dichlorosalicylic acid to solvent is between 0.5:1 and 0.5:2.

6. A process according to claim 1, wherein the crude 3,6-dichlorosalicylic acid and solvent are stirred together for between 30 minutes to 2 hours before filtration.

7. A process according to claim 1, wherein the crude 3,6-dichlorosalicylic acid and solvent are stirred together at a temperature between 35° C. and 60° C.
